(19) United States
(12) Patent Application Publication
(10) Pub. No.: US 2007/0134165 A1
(43) Pub. Date: Jun. 14, 2007

(54) Use of Ciclesonide for the Treatment of Respiratory Disease in a Smoking Patient

(75) Inventors: Wilhelm Wurst, Konstanz (DE); Thomas Bethke, Konstanz (DE); Renate Engelstaetter, Allensbach (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/578,294

(22) PCT Filed: Apr. 19, 2005
(86) PCT No.: PCT/EP05/51718

§ 371 Date: Nov. 2, 2006

Related U.S. Application Data
(60) Provisional application No. 60/563,464, filed on Apr. 20, 2004.

(51) Int. Cl. A61K 31/58; A61K 9/12

(52) U.S. Cl. 424/45; 514/174

(57) ABSTRACT

The invention relates to a new method of treatment of respiratory diseases, in particular the treatment of asthmatic smoking patients. The method comprises the administration of a pharmaceutical composition comprising ciclesonide.

### FIELD OF THE INVENTION

This invention relates to a new method of treatment of respiratory diseases, in particular the treatment of smoking asthmatic patients.

### BACKGROUND

Inhaled corticosteroids (ICS) have been recommended as the most potent and most effective primary maintenance therapy for persistent asthma of all severity grades.

U.S. Pat. No. 5,482,934 discloses pregna-1,4-diene-3,20-dione-16-17-acetal-21 esters and their use in the treatment of inflammatory conditions. The compounds have the general structure: 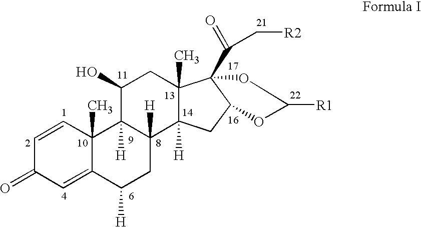
wherein R1 is 2-propyl, 1-butyl, 2-butyl, cyclohexyl or phenyl; and R2 is acetyl or isobutanoyl. Ciclesonide is the INN for a compound of formula I in which R1 is cyclohexyl and R2 is isobutanoyl with the chemical name [11β,16α(R)]-16,17-[(Cyclohexylmethylene)bis(oxy)]-11-hydroxy-21-(2-methyl-1-oxo-propoxy)pregna-1,4-diene-3,20-dione.

Ciclesonide is a novel inhaled corticosteroid for asthma treatment, which is undergoing clinical evaluation. Ciclesonide has very low affinity for the glucocorticosteroid receptor but is readily converted to the active metabolite desisobutyryl-ciclesonide by esterases in the lung to provide local activity in the target organ. This activation occurs by ester cleavage at the C21 position of ciclesonide. The affinity of desisobutyryl-ciclesonide to the glucocorticosteroid receptor is approximately 100 times higher than that of ciclesonide. Ciclesonide is only moderately absorbed after oral administration and has low systemic activity. Concentration of the drug in the lungs is high and metabolism by liver oxidases is very high, giving the drug a low plasma half-life. Systemic activity of ciclesonide is three times lower than that of budesonide, but anti-inflammatory activity is higher for the former.

Pederson et al (Am J Respir Crit Care Med 1996; 153:1519-29) report on a considerable resistance to inhaled steroids in smoking asthmatic subjects found in a one year trial with inhaled budesonide.

Biberger et al (ATS 2003 Seattle May 16-21, 2003 Poster Efficacy and Safety of Ciclesonide compared with budesonide in Asthma Patients: A Randomized 12-Week Study) report on a clinical study to investigate efficacy, safety and onset of action of ciclesonide and budesonide administered once daily in the evening to asthma patients. It is reported that in a subgroup analysis of FEV₁ a trend for superiority of ciclesonide over budesonide was found in ex-smokers.

### SUMMARY OF THE INVENTION

It has now been found surprisingly with regard to the findings reported by Pederson et al that respiratory diseases in smoking patients may be very effectively and safely treated by administering a therapeutically effective and pharmacologically tolerable amount of ciclesonide to the patients. As compared to the treatment with budesonide superior improvements in lung function were observed for smoking asthmatic patients when treated with ciclesonide.

Subject of the invention is therefore a method for treating a respiratory disease in a patient, which patient is a smoking patient and the method comprising administering to the patient a therapeutically effective and pharmacologically tolerable dose of a composition containing ciclesonide, a pharmaceutically acceptable salt, solvates or physiologically functional derivative thereof.

Ciclesonide (herein also referred to as active ingredient) is the INN for an active compound having the chemical name [11β,16α-(R)]-16,17-[(cyclohexylmethylene)bis(oxy)]-11-hydroxy-21-(2-methyl-1-oxopropoxy)pregna-1,4-diene-3,20dione. Ciclesonide and its preparation are described in U.S. Pat. No. 5,482,934. According to the invention, the name ciclesonide also includes solvates of ciclesonide, physiologically functional derivatives of ciclesonide or solvates thereof. Physiologically functional derivatives of ciclesonide, which can be mentioned in connection with the present invention, are preferably chemical derivatives of ciclesonide, which have a similar physiological function as ciclesonide or an active metabolite of ciclesonide, for example the 21-hydroxy derivative of ciclesonide (hereinafter also referred to as desisobutyryl-ciclesonide=des-CIC). The 21-hydroxy compound has the chemical name 16α,17-(22R,S)-cyclohexylmethylenedioxy-11β,21-dihydroxypregna-1,4-diene-3,20-dione. This compound and its preparation are disclosed In WO 94/22899. According to the invention, the name “ciclesonide” is understood as meaning not only the pure R epimer of the compound [11β,16α]16,17-[(cyclohexylmethylene)bis(oxy)]-11-hydroxy-21-(2-methyl-1-oxopropoxy)pregna-1,4-diene-3,20-dione but also R/S epimer mixtures in any desired mixing ratio (that is the compounds [11β,16α(R)]-16,17-[(cyclohexylmethylene)bis(oxy)]-11-hydroxy-21-(2-methyl-1-oxopropoxy)pregna-1,4diene3,20-dione and [11β,16α(S)]-16,17-[(cyclohexylmethylene)bis(oxy)]-11-hydroxy-21-(2-methyl1-oxopropoxy)pregna-1,4-diene-3,20-dione), those being preferred which essentially consist of R epimers. According to the invention, essentially consisting of R epimers means that the proportion of S epimers in the mixture is less than or equal to 5%, preferably less than or equal to 1%.

In connection with the invention ciclesonide is preferably administered to the patient at a daily dose range of from 20 to 1600 μg. Exemplary doses in connection with the invention comprise 20, 40, 60, 80, 100, 120, 140, 160, 180, 200, 320 μg ciclesonide. Preferably the dose comprises 40, 80, 160 or 320 μg ciclesonide. The dose is preferably a daily dose and administered once or twice daily, preferably once daily. A once daily dose may be administered any time of the day, e.g. in the morning or preferably in the evening. The administration of a daily dose of ciclesonide in the range of from 20 to 320 μg is preferably part of a continuous treatment regimen, preferably a treatment period of more than one day, particularly preferably more than one week, e.g. a two week treatment period, a one month treatment period, a one year treatment period or a life long treatment period.

The patient in connection with the invention is a smoker. Smoker in connection with the invention preferably refers to a patient with a smoking history of less than 10 pack per year of cigarettes or less than two pipe packs per years. Smoker in connection with the invention also refers to a patient, which has quit smoking (ex-smoker). In a preferred embodiment ex-smokers refers to a smoker with a smoking history of less than 10 pack per year of cigarettes or less than two pipe packs per years and at least three months of smoking abstinence.

Ciclesonide has been described for use in the treatment of respiratory diseases. Therefore, formulations of ciclesonide have use in the prophylaxis and treatment of clinical conditions for which a glucocorticosteroid is indicated. Such conditions include diseases associated with reversible airways obstruction such as asthma, nocturnal asthma, exercise-induced asthma, chronic obstructive pulmonary diseases (COPD) (e.g. chronic and wheezy bronchitis, emphysema), respiratory tract infection and upper respiratory tract disease (e.g. rhinitis, such as allergic and seasonal rhinitis). In a preferred embodiment according to the Invention the respiratory disease in connection with the invention refers to asthma or chronic obstructive pulmonary disease. Asthma in connection with invention preferably refers to mild to severe asthma/persistent asthma.

The compositions comprising ciclesonide (also referred to as formulations) include those suitable for oral, parenteral including subcutaneous, intradermal, intramuscular, intravenous and intraarticular, intranasal, inhalation (including fine particle dusts or mists which may be generated by means of various types of metered dose pressurized aerosols, nebulisers or insufflators), rectal and topical (including dermal, buccal, sublingual and intraocular administration) although the most suitable route may depend upon for example the condition and disorder of the recipient. The formulations may conveniently be presented in unit dosage form and may be prepared by any of the methods well known in the art of pharmacy. All methods include the step of bringing the active ingredients into association with the carrier, which constitutes one or more accessory ingredients/excipients. In general the formulations are prepared by uniformly and intimately bringing into association the active ingredient with liquid carriers or finely divided solid carriers or both and then, if necessary, shaping the product into the de sired formulation.

In one embodiment ciclesonide is provided in a form suitable for Inhalation. Formulations for inhalation include powder compositions, which will preferably contain lactose, and spray compositions which may be formulated, for example, as aqueous solutions or suspensions or as aerosols delivered from pressurized packs, with the use of a suitable propellant, e. g. 1,1,1,2-terafluorethane, 1,1,1,2,3,3,3-heptafluoropropane, carbon dioxide or other suitable gas. A class of propellants, which are believed to have minimal ozone-depleting effects in comparison to conventional chlorofluorocarbons comprise hydrofluorocarbons and a number of medicinal aerosol formulations using such propellant systems are disclosed in, for example, EP 0372777, WO91/04011, WO91/11173, WO91/11495, WO91/14422, WO93/11743, and EP0553298. These applications are all concerned with the preparation of pressurized aerosols for the administration of medicaments and seek to overcome problems associated with the use of this new class of propellants, in particular the problems of stability associated with the pharmaceutical formulations prepared. The applications propose, for example, the addition of one or more of excipients such as polar cosolvents or wetting agents (e.g. alcohols such as ethanol), alkanes, dimethyl ether, surfactants (including fluorinated and non-fluorinated surfactants, carboxylic acids such as oleic acid, polyethoxylates etc.) or bulking agents such as a sugar (see for example WO02/30394) and vehicles such as cromoglicic acid and/or nedocromil which are contained at concentrations, which are not therapeutically and prophylactically active (see WO00/07567). For suspension aerosols, the active ingredients should be micronised so as to permit inhalation of substantially all of the active ingredients into the lungs upon administration of the aerosol formulation, thus the active ingredients will have a mean particle size of less than 100 microns, desirably less than 20 microns, and preferably in the range 0.7 to 10 microns, for example, 1 to 5 microns.

WO 98/52542 is related to pharmaceutical compositions comprising a therapeutically effective amount of ciclesonide or a related compound and a hydrofluorocarbon propellant, preferably selected from 1,1,1,2-tetafluoroethane, 1,1,1,2,3,3,3heptafluoropropane and a mixture thereof, and cosolvent, preferably ethanol, in an amount effective to solubilize ciclesonide and optionally a surfactant. In a preferred embodiment ciclesonide Is administered in a composition according to WO98/52542. In contrast to traditional suspension or powder formulation such solution formulation provides a fine particle spray, yielding high pulmonary deposition in central and peripheral regions.

Ciclesonide is generally present in the formulation at a concentration, which allows administration of a dose of from 20 to 1600 μg. Such formulation generally comprises ethanol in an amount effective to solubilize the ciclesonide. The propellant preferably includes a hydrofluoroalkane, in particular Propellant 134a, Propellant 227 or a mixture thereof. In the case of a mixture the ratio of Propellant 134a to Propellant 227 is generally in a range from 75:25 w/w to 25:75 w/w. The formulations may contain surfactant such as oleic acid, but may be also free of surfactant. The formulations are preferably free of other excipients.

The formulations may be manufactured by preparing a drug concentrate of the active ingredients with ethanol and adding this concentrate to the pre-chilled propellant in a batching vessel. Preferably a solution of the ciclesonide in the cosolvent is added to the prechilled propellant in a batching vessel. The resulting formulation is filled into vials. Alternatively the formulations may be prepared by adding the required quantity of active ingredient into an aerosol vial, crimping a valve on the vial and introducing a pre-mixed blend of propellant and ethanol through the valve. The vial is placed in an ultra-sonic bath to ensure solubilization of ciclesonide.

In another embodiment preferred compositions for aerosol delivery contain the active ingredient in particulate form, and 1,1,1,2-tetrafluoroethane, 1,1,1,2,3,3,3heptafluoropropane or mixtures thereof as propellant. Such formulation generally comprises from 0.01 to 5% (w/w relative to the total weight of the formulation) of polar cosolvent, in particular ethanol. In a preferred embodiment no or less than 3% w/w of polar cosolvent, in particular ethanol is contained. Especially preferred compositions for aerosol delivery consist of particulate active ingredient, and 1,1,1,2-tetrafluoroethane, 1,1,1,2,3,3,3-heptafluorpropane or mixtures thereof as propellant and optionally a surfactant (preferably oleic acid). In the case of a mixture the ratio of Propellant 134a to Propellant 227 is generally in a range from 75:25 w/w to 25:75 w/w.

The formulations may be prepared by adding the required quantity of active ingredient into an aerosol vial, crimping a valve on the vial and introducing propellant or optionally a pre-mixed blend of propellant and optionally the cosolvent and surfactant through the valve.

Canisters generally comprise a container capable of withstanding the vapor pressure of the propellant, such as plastic or plastic-coated glass bottle or a metal can, for example an aluminium can which may optionally be anodized, lacquer-coated and/or plastic-coated, which container is closed with a metering valve. Canisters may be coated with a fluorocarbon polymer as described in WO 96/32150, for example, a co-polymer of polyethersulphone (PES) and polytetrafluoroethylene (PTFE). Another polymer for coating that may be contemplated is FEP (fluorinated ethylene propylene).

The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in WO92/11190 and valves containing EPDM rubber as described in WO95/02650 may be suitable. Suitable valves are commercially available from manufacturers well known in the aerosol industry, for example, from Valois, France (e.g. DF10, DF30, DF60), Bespak pic, UK (e.g. BK300, BK356, BK357) and 3M-Neotechnic Ltd., UK (e.g. Spraymiser).

Valve seals, especially the gasket seal and also the seals around the metering chamber, can be manufactured of a material, which is inert to and resists extraction into the contents of the formulation, especially when the contents include ethanol.

Valve materials, especially the material of manufacture of the metering chamber, can be manufactured of a material, which is inert to and resists distortion by contents of the formulation, especially when the contents include ethanol. Particularly suitable materials for use in manufacture of the metering chamber include polyesters e.g. polybutyleneterephthalate (PBT) and acetals, especially PBT.

Materials of manufacture of the metering chamber and/or the valve stem may desirably be fluorinated, partially fluorinated or impregnated with fluorine containing substances in order to resist drug deposition.

Valves, which are entirely or substantially composed of metal components (e.g. Spraymiser, 3M-Neotechnic), are especially preferred for use according to the invention.

Intranasal sprays or nasal drops may be formulated with aqueous or non-aqueous vehicles with or without the addition of agents such as thickening agents, buffer salts or acid or alkali to adjust the pH, isotonicity adjusting agents, preservatives or anti-oxidants. Suitable aqueous formulations for ciclesonide for application to mucosa are for example disclosed in WO01/28562 and WO01/28563.

In another embodiment of the invention the pharmaceutical formulation comprising the ciclesonide in as a dry powder, i.e. ciclesonide is present in a dry powder comprising finely divided ciclesonide optionally together with a finely divided pharmaceutically acceptable carrier, which is preferably present and may be one or more materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose, particularly in the form of the monohydrate. The dry powder may be in capsules of gelatine or plastic, or in blisters, for use in a dry powder inhalation device, preferably in dosage units of the ciclesonide together with the carrier in amounts to bring the total weight of powder in each capsule to from 5 mg to 50 mg. Alternatively the dry powder may be contained in a reservoir of a multi-dose dry powder inhalation device. Capsules and cartridges of for example gelatin, or blisters of for example laminated aluminium foil, for use in an inhaler or insulator may be formulated containing a powder mix of the active ingredients and a suitable powder base such as lactose or starch, preferably lactose. In this aspect the active ingredient is suitably micronised so as to permit inhalation of substantially all of the active ingredients into the lungs upon administration of the dry powder formulation, thus the active ingredient will have a particle size of less than 100 μm, desirably less than 20 μm, and preferably in the range 1 to 10 μm. The solid carrier, where present, generally has a maximum particle diameter of 300 μm, preferably 200 μm, and conveniently has a mean particle diameter of 40 to 100 μm, preferably 50 to 75 μm. The particle size of the active ingredient and that of a solid carrier where present in dry powder compositions, can be reduced to the desired level by conventional methods, for example by grinding in an air-jet mill, ball mill or vibrator mill, microprecipitation, spray drying, lyophilisaton or recrystallisation from supercritical media.

Where the inhalable form of the composition of the invention is the finely divided particulate form, the inhalation device may be, for example a dry powder inhalation device adapted to deliver dry powder from a capsule or blister containing a dosage unit of the dry powder or a multi-dose dry powder inhalation device. Such dry powder inhalation devices are known in the art. Examples which may be mentioned are Cyclohaler®, Diskhaler® Rotadisk®, Turbohaler® or the dry powder inhalation devices disclosed EP 0 505 321, EP 407028, EP 650410, EP 691865 or EP 725725 (Ultrahaler®).

Formulations for inhalation by nebulization may be formulated with an aqueous vehicle with the addition of agents such as acid or alkali, buffer salts, isotonicity adjusting agents or antimicrobials. They may be sterilized by filtration or heating in an autoclave. Suitable technologies for this type of administration are known in the art. As an example the Mystic® technology is to be mentioned (see for example U.S. Pat. No. 6,397,838, U.S. Pat. No. 6,454,193 and U.S. Pat. No. 6,302,331).

Preferred unit dosage formulations are those containing a pharmaceutical effective dose, as hereinbefore recited, or an appropriate fraction thereof, of the active ingredient. Thus, in the case of formulations designed for delivery by metered dose pressurized aerosols, one actuation of the aerosol may deliver half of the therapeutical effective amount such that two actuations are necessary to deliver the therapeutically effective dose.

Further subject of the invention is the use of ciclesonide for the manufacture of a medicament for the treatment of a respiratory disease in a smoking patient.

It should be understood that in addition to the ingredients particularly mentioned above, the formulations of this invention may include other agents conventional in the art having regard to the type of formulation in question. Furthermore, the claimed formulations include bioequivalents as defined by the US Food and Drugs Agency.

Although the invention has been described in terms of preferred formulations and ingredients, it will be understood that these are not intended to be limiting. To the contrary, those skilled in the art will understand that various optional ingredients may be included, such as flavoring agents, preservatives, additional active ingredients, and the like, while still embodying the present invention.

The invention will now be illustrated by the following examples without restricting it.

### EXAMPLES

### Example 1

### Ciclesonide Metered Dose Inhaler (HFA-MDI)

Ciclesonide is provided as pharmaceutical product comprising an aerosol vial equipped with a dispensing valve and containing the following formulation:

| | |
|---|---|
| Ciclesonide | 1.000 mg/ml |
| Ethanol | 94.800 mg/ml |
| P134a | 1090.200 mg/ml |

### Example 2

### Clinical Study

This was a double-blind, randomized, parallel group study during which patients with asthma received ciclesonide 200 μg (metered dose inhaler with ciclesonide 100 μg/puff) once daily in the evening or budesonide 400 μg (a Turbuhaler® with budesonide 200 μg/puff) once daily in the evening for 12 weeks. The objectives were to compare the effect of 200 μg ciclesonide with 400 μg budesonide once daily in the evening for 12 weeks, on pulmonary function, asthma symptoms and use of rescue medication. Another objective was to provide information on the safety and tolerability of ciclesonide.

Efficacy Results:

After 12 weeks of treatment, no statistically significant within-treatment difference was observed for the primary variable FEV₁ (forced expiratory volume in one second) or the co-primary variables FVC (forced vital capacity) and morning PEF (peak expiratory flow) in patients of both treatment groups. In contrast, subgroup analyses revealed distinct differences in favor of ciclesonide compared to the results of the overall population. In the primary variable FEV1, statistically significant increases were seen in the ciclesonide group in patients that were (ex-)smokers. In none of the corresponding subgroup within the budesonide group statistically significant changes in FEV1 were observed.

TABLE 1
Shows the effect on ex-smokers
(CIC refers to the ciclesonide treatment,
BUD refers to the budesonide treatment).
ex smokers, ITT, al values as LSmeans

| | CIC | | BUD | |
|---|---|---|---|---|
| | start | end | start | end |
| FEV1 (L) | 2.336 | 2.773 | 2.339 | 2.602 |
| FVC (L) | 3.146 | 3.604 | 3.146 | 3.385 |
| PEF (L/min) | 310 | 391 | 310 | 357 |
| morning PEF (L/mm) | 370 | 416 | 370 | 391 |

1. A method for treating a respiratory disease in a patient, wherein said patient is a smoking patient, comprising administering to the patient a therapeutically effective and pharmacologically tolerable dose of a composition containing ciclesonide, or a pharmaceutically acceptable salt, solvate or physiologically functional derivative thereof.

2. The method according to claim 1, wherein the dose comprises 20, 40, 60, 80, 100, 120, 140, 160, 180, 200 or 320 μg ciclesonide.

3. The method according to claim 1, wherein the dose is a daily dose in a continuous treatment regimen.

4. The method according to claim 3, wherein the treatment period is more than one day.

5. The method according to claim 4, wherein the treatment period is more than one week.

6. The method according to claim 5, wherein the composition comprises a pharmaceutically acceptable carrier and/or one or more excipients.

7. The method according to claim 1, wherein ciclesonide is selected from the group consisting of [11β,16α(R)]-16,17-[(Cyclohexylmethylene)bis(oxy)]-11-hydroxy-21-(2-methyl-1-oxopropoxy)pregna-1,4-diene-3,20-dione, [11β,16α(S)]-16,17-[(Cyclohexylmethylene)bis(oxy)]-11-hydroxy-21-(2-methyl-1-oxoprop-oxy)pregna-1,4-diene3,20-dione, [11β,16α(R,S)]-16,17-[(Cyclohexylmethylene)bis(oxy)]-11-hydroxy-21-(2-methyl-1-oxoprop-oxy)pregna-1,4-diene3,20-dione, 16α,17-(22R)-Cyclohexylmethylendioxy-11β,21-dihydroxy-pregna-1,4-dien-3,20-dion, 16α,17-(22S)-Cyclohexylmethylendioxy-11β,21-dihydroxy-pregna-1,4-dien-3,20-dion and 16α,17-(22R,S)-Cyclohexylmethylendioxy-11β,21-dihydroxy-pregna-1,4-dien-3,20-dion.

8. The method according to claim 1, comprising a once daily dosage regimen.

9. The method according to claim 1, wherein the composition is suitable for administration by inhalation.

10. The method according to claim 9, wherein the composition comprises ciclesonide dissolved in a pharmaceutically acceptable carrier.

11. The method according to claim 10, wherein the composition is a pharmaceutical aerosol formulation comprising a therapeutically effective amount of ciclesonide and a hydrofluorocarbon propellant, and cosolvent in an amount effective to solubilize ciclesonide and optionally a surfactant.

12. The method according to claim 11, wherein the cosolvent is ethanol.

13. The method according to claim 12, wherein the composition is a pharmaceutical aerosol formulation comprising particles of ciclesonide in a therapeutically effective amount and a hydrofluorocarbon propellant, and 0.01 to 5% w/w based upon propellant of polar cosolvent and optionally a surfactant.

14. The method according to claim 9, wherein the composition is a dry powder and the carrier is a saccharide.

15. The method according to claim 14, wherein the carrier is lactose monohydrate.

16. The method according to claim 15, wherein the respiratory disease is selected from the group consisting of asthma, nocturnal asthma, exercise-induced asthma, chronic obstructive pulmonary diseases (COPD), chronic bronchitis, wheezy bronchitis, emphysema, respiratory tract infection, upper respiratory tract disease, rhinitis, allergic rhinitis and seasonal rhinitis.

17. The method according to claim 1, wherein the respiratory disease is mild or moderate asthma.

18. The method according to claim 1, wherein the ciclesonide consists essentially of R epimer.

19. The method according to claim 1, wherein the patient has a smoking history of less than 10 pack per year of cigarettes or less than two pipe packs per year.

20. The method according to claim 19, wherein the patient has a smoking history of less than 10 pack per year of cigarettes or less than two pipe packs per year and has at least three months of smoking abstinence.

21. The method of claim 11, wherein the hydrofluorocarbon propellant is selected from the group consisting of 1,1,1,2-tetrafluoroethane, 1,1,1,2,3,3,3-heptafluoropropane and a mixture thereof.

22. The method of claim 13, wherein the hydrofluorocarbon propellant is selected from the group consisting of 1,1,1,2-tetrafluoroethane, 1,1,1,2,3,3,3-heptafluoropropane and a mixture thereof.
